(19) United States
(12) Patent Application Publication
(10) Pub. No.: US 2015/0247171 A1
(43) Pub. Date: Sep. 3, 2015

(54) Carbon Capture in Fermentation

(71) Applicant: LANZATECH NEW ZEALAND LIMITED, Parnell, Auckland (NZ)

(72) Inventors: Michael Anthony Schultz, Roselle, IL (US); Derek Wayne Griffin, Roselle, IL (US)

(73) Assignee: LanzaTech New Zealand Limited, Auckland (NZ)

(21) Appl. No.: 13/818,854

(22) PCT Filed: Feb. 7, 2013
(86) PCT No.: PCT/US2013/025218

§ 371 Date: Feb. 25, 2013

Related U.S. Application Data
(60) Provisional application No. 61/597,122, filed on Feb. 9, 2012.

(51) Int. Cl. C12P 7/54; C12P 7/06

(52) U.S. Cl. CPC C12P 7/54; C12P 7/065

(57) ABSTRACT

The present invention provides methods and systems for improving carbon capture from a gas stream comprising methane. Further, the invention provides a method for the production of at least one alcohol, and at least one acid from a gas stream comprising methane, the method comprising reforming a gas stream comprising methane to provide a syngas, in a first bioreactor fermenting the syngas to produce at least one acid and a tail gas comprising CO₂ and H₂, and, in a second bioreactor fermenting the tail gas to produce at least one acid.

### CROSS REFERENCE TO RELATED APPLICATIONS

This application is a continuation of International Application PCT/US2013/025218 filed on Feb. 7, 2013 which claims the benefit of U.S. Provisional Application No. 61/597,122 filed on Feb. 9, 2012 both of which are incorporated by reference in their entirety.

### FIELD OF THE INVENTION

This invention relates to a method for improving carbon capture from a natural gas stream. More particularly the invention relates to a method for improving carbon capture from a natural gas stream including a natural gas reforming step for producing a syngas stream, an alcohol fermentation step for producing one or more alcohols and a gaseous by-product, and an acid fermentation step for producing one or more acids.

### BACKGROUND OF THE INVENTION

Ethanol is rapidly becoming a major hydrogen-rich liquid transport fuel around the world. Worldwide consumption of ethanol in 2002 was an estimated 10.8 billion gallons. The global market for the fuel ethanol industry has also been predicted to grow sharply in future, due to an increased interest in ethanol in Europe, Japan, the USA and several developing nations.

For example, in the USA, ethanol is used to produce E10, a 10% mixture of ethanol in gasoline. In E10 blends the ethanol component acts as an oxygenating agent, improving the efficiency of combustion and reducing the production of air pollutants. In Brazil, ethanol satisfies approximately 30% of the transport fuel demand, as both an oxygenating agent blended in gasoline, or as a pure fuel in its own right. Also, in Europe, environmental concerns surrounding the consequences of Green House Gas (GHG) emissions have been the stimulus for the European Union (EU) to set member nations a mandated target for the consumption of sustainable transport fuels such as biomass derived ethanol.

The vast majority of fuel ethanol is produced via traditional yeast-based fermentation processes that use crop derived carbohydrates, such as sucrose extracted from sugarcane or starch extracted from grain crops, as the main carbon source. However, the cost of these carbohydrate feed stocks is influenced by their value as human food or animal feed, while the cultivation of starch or sucrose-producing crops for ethanol production is not economically sustainable in all geographies. Therefore, it is of interest to develop technologies to convert lower cost and/or more abundant carbon resources into fuel ethanol.

CO is a major low cost energy-rich by-product of the incomplete combustion of organic materials such as coal or oil and oil derived products. For example, the steel industry in Australia is reported to produce and release into the atmosphere over 500,000 tonnes of CO annually.

It has long been recognised that catalytic processes may be used to convert gases consisting primarily of CO and/or CO and hydrogen (H₂) into a variety of fuels and chemicals. However, micro-organisms may also be used to convert these gases into fuels and chemicals. These biological processes, although generally slower than chemical reactions, have several advantages over catalytic processes, including higher specificity, higher yields, lower energy costs and greater resistance to poisoning.

The ability of micro-organisms to grow on CO as their sole carbon source was first discovered in 1903. This was later determined to be a property of organisms that use the acetyl coenzyme A (acetyl CoA) biochemical pathway of autotrophic growth (also known as the Woods-Ljungdahl pathway and the carbon monoxide dehydrogenase/acetyl CoA synthase (CODH/ACS) pathway). A large number of anaerobic organisms including carboxydotrophic, photosynthetic, methanogenic and acetogenic organisms have been shown to metabolize CO to various end products, namely CO₂, H₂, methane, n-butanol, acetate and ethanol. While using CO as the sole carbon source all such organisms produce at least two of these end products.

Anaerobic bacteria, such as those from the genus Clostridium, have been demonstrated to produce ethanol from CO, CO₂ and H₂ via the acetyl CoA biochemical pathway. For example, various strains of Clostridium ljungdahlii that produce ethanol from gases are described in WO 00/68407, EP 117309, U.S. Pat. Nos. 5,173,429, 5,593,886, and 6,368,819, WO 98/00558 and WO 02/08438. The bacterium Clostridium autoethanogenum sp is also known to produce ethanol from gases (Aribini et al, Archives of Microbiology 161, pp 345-351 (1994)).

However, ethanol production by micro-organisms by fermentation of gases is always associated with co-production of acetate and/or acetic acid. As some of the available carbon is converted into acetate/acetic acid rather than ethanol, the efficiency of production of ethanol using such fermentation processes may be less than desirable. Also, unless the acetate/acetic acid by-product can be used for some other purpose, it may pose a waste disposal problem. Acetate/acetic acid is converted to methane by micro-organisms and therefore has the potential to contribute to Green House Gas emissions.

The importance of controlling parameters of the liquid nutrient medium used for culturing bacteria or micro-organisms within a bioreactor used for fermentation has been recognised in the art. NZ 556615, filed 18 Jul. 2007 and incorporated herein by reference, describes, in particular, manipulation of the pH and the redox potential of such a liquid nutrient medium. For example, in the culture of anaerobic acetogenic bacteria, by elevating the pH of the culture to above about 5.7 while maintaining the redox potential of the culture at a low level (−400 mV or below), the bacteria convert acetate produced as a by-product of fermentation to ethanol at a much higher rate than under lower pH conditions. NZ 556615 further recognises that different pH levels and redox potentials may be used to optimise conditions depending on the primary role the bacteria are performing (i.e., growing, producing ethanol from acetate and a gaseous CO-containing substrate, or producing ethanol from a gaseous containing substrate).

U.S. Pat. No. 7,078,201 and WO 02/08438 also describe improving fermentation processes for producing ethanol by varying conditions (e.g. pH and redox potential) of the liquid nutrient medium in which the fermentation is performed.

The pH of the liquid nutrient medium may be adjusted by adding one or more pH adjusting agents or buffers to the medium. For example, bases such as NaOH and acids such as sulphuric acid may be used to increase or decrease the pH as required. The redox potential may be adjusted by adding one or more reducing agents (e.g. methyl viologen) or oxidising agents.

Similar processes may be used to produce other alcohols, such as butanol, as would be apparent to one of skill in the art.

Regardless of the source used to feed the fermentation reaction, problems can occur when there are breaks in the supply. More particularly, such interruptions can be detrimental to the efficiency of the micro-organisms used in the reaction, and in some cases, can be harmful thereto.

For example, where CO gas in an industrial waste gas stream is used in fermentation reactions to produce acids/alcohols, there may be times when the stream is not produced. During such times, the micro-organisms used in the reaction may go into hibernation. When the stream is available again, there may then be a lag before the micro-organisms are fully productive at performing the desired reaction.

### SUMMARY OF THE INVENTION

According to the invention, there is provided a method for improving carbon capture in a fermentation process.

In a first aspect there is provided a method for producing at least one alcohol and at least one acid from a gas stream comprising methane, the method comprising;
a. Flowing the gas stream to a reforming module and reforming the gas stream to produce a syngas substrate comprising CO, CO₂ and H₂;
b. Flowing the syngas substrate to a first bioreactor, the first bioreactor comprising a liquid nutrient media comprising a culture of one or more carboxydotrophic micro-organisms;
c. Fermenting the syngas substrate to produce at least one alcohol and a tail gas stream comprising H₂ and CO₂;
d. Flowing the tail gas stream to a second bioreactor, the second bioreactor comprising a liquid nutrient medium comprising a culture of one or more microorganism; and
e. Fermenting the tail gas stream to produce one or more acids.

In one embodiment of the invention the composition of the tail gas stream exiting the first bioreactor is controlled at a desired ratio of H₂:CO₂ by measuring the amount of CO and H₂ consumed by the one or more carboxydotrophic microorganism and adjusting the syngas substrate in response to changes in the amount of CO and H₂ consumed.

In a second aspect there is provided a method for improving carbon capture from al gas stream comprising methane, the method comprising;
a. receiving the gas stream;
b. passing the gas stream to a reformer;
c. reforming the gas stream to produce a syngas comprising CO, CO₂ and H₂;
d. passing the syngas to a bioreactor containing a culture of one or more microorganisms;
e. fermenting the syngas to produce one or more alcohol(s) and a tail gas stream comprising CO₂ and H₂;
f. passing the tail gas stream to a second bioreactor containing a culture of one or more microorganisms;
g. fermenting the tail gas stream to produce one or more acids.

In one embodiment the gas reforming module is selected from the group comprising; dry reforming, steam reforming, partial oxidation, and auto thermal reforming.

In one embodiment, the reforming module can also be followed by a water gas shift reaction or a reverse water gas shift reaction. According to certain embodiments of the invention, the syngas produced by the reforming module has a H2:CO ratio of 1:1; or 2:1; or 3:1; or 4:1; or at least 5:1.

In one embodiment of the invention, the syngas produced by the gas reforming reactions further comprises sulfur components and other contaminants.

In one embodiment of the invention, the fermentation of syngas to ethanol utilises CO and optionally H₂. In certain embodiments, little or no hydrogen is used in the fermentation reaction. In certain embodiment, in particular in syngas streams where CO supply is limited, hydrogen is used in the fermentation reaction.

In one embodiment, the composition of the syngas provided to the first bioreactor is controlled such that the tail gas exiting the first bioreactor has a desired H2:CO2 ratio. In one embodiment of the invention, the uptake of H₂ and CO by the culture in the first bioreactor is monitored, and the composition of the gas introduced to the first bioreactor is adjusted to provide a tail gas having the desired H₂:CO₂ ratio.

In one embodiment of the invention, the one or more alcohol(s) is selected from the group comprising ethanol, propanol, butanol and 2,3-butanediol. In particular embodiments the one or more alcohol(s) is ethanol. In one embodiment the one or more acid(s) is acetic acid.

In one embodiment of the invention the tail gas exiting the primary bioreactor is rich in CO₂ and H₂.

In one embodiment of the invention the tail gas exiting the primary bioreactor is passed into a secondary bioreactor for fermentation. In accordance with one embodiment, the CO₂ and H₂ are converted to acetic acid during the fermentation process in the secondary bioreactor.

In one embodiment of the invention, tail gas exiting the primary bioreactor comprises H₂ and CO₂ at a ratio of at least 1:1 or at least 2:1 or at least 3:1. In alternative embodiments the tail gas exiting the bioreactor is blended with H₂ and/or CO₂ to provide a gas stream with a desired 2:1 H₂:CO₂ ratio. In certain embodiments excess H₂ and/or CO₂ is removed from the tail gas exiting the bioreactor to provide a gas stream with a desired H₂:CO₂ ratio of 2:1

In one embodiment the gas stream comprising methane is selected from the group consisting of: natural gas, methane sources including coal bed methane, stranded natural gas, landfill gas, synthetic natural gas, natural gas hydrates, methane produced form catalytic cracking of olefins or organic matter, and methane produces as an unwanted byproduct from CO hydrogenation and hydrogenolysis reactions such as the Fischer-Tropsch process.

In one embodiment the gas stream comprising methane is a natural gas stream.

In accordance with a third aspect of the invention, there is provided a method for improving carbon capture from a gas stream comprising methane, the method comprising;
a. reforming the gas stream to produce a syngas stream;
b. passing the syngas stream to a hydrogen separation module, wherein at least a portion of the hydrogen is removed from the syngas stream;
c. passing the hydrogen depleted syngas stream to a primary bioreactor containing a culture of one or more microorganisms;
d. fermenting the syngas to produce one or more alcohols;
e. passing a tail gas produced as a by product of the fermentation reaction of (d) to a secondary bioreactor containing a culture of one or more microorganism;
f. Fermenting the tail gas to produce one or more acids.

In one embodiment of the invention, the reformed syngas stream is rich in hydrogen. In one embodiment of the invention at least a portion of the hydrogen separated from the syngas stream in the hydrogen separation module is passed to a secondary bioreactor, for fermentation to one or more acid(s).

In certain embodiments, excess hydrogen separated from the syngas stream is collected, or directed to another process.

In one embodiment, the fermentation on the primary bioreactor is controlled such that the uptake of hydrogen by the culture is minimised.

In one embodiment of the invention, tail gas exiting the primary bioreactor comprises H₂ and CO₂ at a ratio of at least 1:1 or at least 2:1 or at least 3:1. In alternative embodiments the tail gas exiting the bioreactor is blended with H₂ and/or CO₂ to provide a gas stream with a desired 2:1 H₂:CO₂ ratio. In certain embodiments excess H₂ and/or CO₂ is removed from the tail gas exiting the bioreactor to provide a gas stream with a desired H₂:CO₂ ratio of 2:1

In accordance with a fourth aspect of the invention there is provided a method for optimising carbon capture of a gas stream comprising methane, the method comprising;
a. reforming a the gas stream to produce a syngas;
b. reacting the syngas in a water gas shift reactor to increase the hydrogen composition of the syngas;
c. fermenting the syngas in a primary bioreactor containing a culture of one or more microorganisms to produce one or more alcohol(s);
d. passing a tail gas comprising CO₂ and H₂ to a second bioreactor containing a culture of one or more microorganisms;
e. fermenting the tail gas to produce one or more acids.

In one embodiment of the invention the water gas shift reaction increases the hydrogen balance of the syngas, such that the hydrogen:CO₂ ratio of the tail gas exiting the primary bioreactor is substantially 2:1.

In one embodiment of the invention, reformed syngas is passed directly into the primary bioreactor, instead of passing through the water gas shift reactor. In accordance with one embodiment, the tail gas exiting the primary bioreactor passes into a water gas shift reactor to increase the hydrogen composition of the tail gas being. The hydrogen enriched tail gas is then passed to the secondary bioreactor.

Although the invention is broadly as defined above, it is not limited thereto and also includes embodiments of which the following description provides examples.

### BRIEF DESCRIPTION OF THE DRAWINGS

The invention will now be described in more detail and with reference to the accompanying figures, in which:

FIG. 1 is an integrated process flow scheme showing co production of ethanol and acetic acid in accordance with one embodiment of the invention.

FIG. 2 is a process flow scheme according to an alternative embodiment of the invention.

FIG. 3 is a flow scheme showing a process alternative wherein the hydrogen content is increased by a water gas shift reaction on reformed syngas.

FIG. 4 is a flow scheme showing a process alternative wherein the hydrogen content of the feed gas to an acid fermentation is increased using a water gas shift reaction.

Table 1 shows the ratio of CO/H2 required in a reformed natural gas stream entering the alcohol fermentation bioreactor to generate a tail-gas exiting the alcohol fermentation with a H₂:CO₂ ratio of 2:1.

### DETAILED DESCRIPTION OF THE INVENTION

### Definitions

Unless otherwise defined, the following terms as used throughout this specification are defined as follows:

The term “substrate comprising carbon monoxide and/or hydrogen” and like terms should be understood to include any substrate in which carbon monoxide and/or hydrogen is available to one or more strains of bacteria for growth and/or fermentation, for example.

“Gaseous substrate comprising carbon monoxide and/or hydrogen” includes any gas which contains carbon monoxide and/or hydrogen. The gaseous substrate may contain a significant proportion of CO, preferably at least about 2% to about 75% CO by volume and/or preferably about 0% to about 95% hydrogen by volume.

“Syngas” includes any gas which contains varying amounts of carbon monoxide and hydrogen. Typically syngas refers to a gas which is produced by reforming or gasification processes. In the context of fermentation products, the term “acid” as used herein includes both carboxylic acids and the associated carboxylate anion, such as the mixture of free acetic acid and acetate present in a fermentation broth as described herein. The ratio of molecular acid to carboxylate in the fermentation broth is dependent upon the pH of the system. The term “acetate” includes both acetate salt alone and a mixture of molecular or free acetic acid and acetate salt, such as the mixture of acetate salt and free acetic acid present in a fermentation broth as may be described herein. The ratio of molecular acetic acid to acetate in the fermentation broth is dependent upon the pH of the system.

The term “hydrocarbon” includes any compound that includes hydrogen and carbon. The term “hydrocarbon” incorporates pure hydrocarbons comprising hydrogen and carbon, as well as impure hydrocarbons and substituted hydrocarbons. Impure hydrocarbons contain carbon and hydrogen atoms bonded to other atoms. Substituted hydrocarbons are formed by replacing at least one hydrogen atom with an atom of another element. The term “hydrocarbon” as used herein includes compounds comprising hydrogen and carbon, and optionally one or more other atoms. The one or more other atoms include, but are not limited to, oxygen, nitrogen and sulfur. Compounds encompassed by the term “hydrocarbon” as used herein include at least acetate/acetic acid; ethanol, propanol, butanol, 2,3-butanediol, butyrate, propionate, caproate, propylene, butadiene, isobutylene, ethylene, gasoline, jet fuel or diesel.

The term “bioreactor” includes a fermentation device consisting of one or more vessels and/or towers or piping arrangements, which includes a Continuous Stirred Tank Reactor (CSTR), Immobilized Cell Reactor (ICR), Trickle Bed Reactor (TBR), Bubble Column, Gas Lift Fermenter, Membrane Reactor such as a Hollow Fibre Membrane Bioreactor (HFMBR), Static Mixer, or other vessel or other device suitable for gas-liquid contact.

Unless the context requires otherwise, the phrases “fermenting”, “fermentation process” or “fermentation reaction” and the like, as used herein, are intended to encompass both the growth phase and product biosynthesis phase of the process. As will be described further herein, in some embodiments the bioreactor may comprise a first growth reactor and a second fermentation reactor. As such, the addition of metals or compositions to a fermentation reaction should be understood to include addition to either or both of these reactors.

“Fermentation broth” is defined as the culture medium in which fermentation occurs.

“A gas stream comprising methane” is defined as any substrate stream comprising CH4 as the main component. This and similar terms include feedstock sources including, but not limited to, natural gas, methane sources including coal bed methane, stranded natural gas, landfill gas, synthetic natural gas, natural gas hydrates, methane produced form catalytic cracking of olefins or organic matter, and methane produces as an unwanted byproduct from CO hydrogenation and hydrogenolysis reactions such as the Fischer-Tropsch process.

The term “natural gas” is used within the specification to exemplify the use of that specific stream. A skilled person would understand that the above mentioned alternative feedstock sources (paragraph [00054]) can be substituted into any or all of the descriptions”.

“Natural gas reforming process” or “gas reforming process” is defined as the general process by which syngas is produced and recovered by a reforming reaction of a natural gas feedstock. The gas reforming process may include any one or more of the following processes;
i) steam reforming processes;
ii) dry reforming processes;
iii) partial oxidation processes;
iv) auto-thermal reforming processes;
v) water gas shift processes; and
vi) reverse water gas shift processes.

The reference herein to gaseous composition percentages are expressed in volume by volume (v/v) terms.

### The Steam Reforming Process

The industrial production of hydrogen using steam reforming of suitable hydrocarbon reactants (primarily methane from natural gas) generally comprises two steps—a steam reforming step and a water-gas shift step. Where methane is referred to herein, it will be appreciated by one of skill in the art that in alternative embodiments of the invention, the steam reforming process may proceed using other suitable hydrocarbon reactants, such as ethanol, methanol, propane, gasoline, autogas and diesel fuel, all of which may have differing reactant ratios and optimal conditions.

In a typical steam reforming process, methane is reacted with steam generally at a stoichiometric excess of steam to carbon in the feed in the presence of a nickel-based catalyst at a pressure of approximately 25 atm and at a temperature of approximately 700-1100° C., more preferably a temperature of approximately 800-900° C., more preferably approximately 850° C. The steam reforming reaction yields carbon monoxide and hydrogen as shown by the following equation:

CH₄+H₂O→CO+3H₂

A typical output gas composition from the steam reforming process would include the following approximate composition: H₂-73%, CO₂-10%, CO-8%, CH₄-4%.

### Partial Oxidation

The reaction of methane with oxygen can be either a non-catalytic reaction at high temperatures (1200-1500° C.), or reaction over a catalyst at lower temperatures. The oxidation of natural gas occurs in an excess of oxygen as follows;

Partial Oxidation CH₄+½O2→CO+2H₂

Full Oxidation CH₄+O₂→CO2+2H₂O

### Dry Reforming

Dry reforming is a catalytic reaction with methane and carbon dioxide over a catalyst at a temperature of 700-800° C. The catalyst is typically a nickel catalyst. The stoichiometry of the reaction is;

CO₂+CH₄→2CO+2H₂

### Auto-Thermal Reforming

Auto-thermal reforming is a combination of steam or CO₂ reforming and partial oxidation, as follows:

2CH₄+O₂+CO₂→3H₂+3CO+H₂O auto-thermal reforming with CO₂

4CH₄+O₂+2H₂O→10H₂+4CO auto-thermal reforming with steam.

In these reactions, steam and/or CO₂ are fed along with oxygen. The exothermic combustion of O₂ can provide heat for the endothermic steam or dry reforming reactions.

### Water Gas Shift Reaction

A water-gas shift (WGS) process may be primarily used to reduce the level of CO in the gas stream received from the steam reforming step and to increase the concentration of H₂. It is envisaged in one embodiment of the invention that the WGS step may be omitted and the gas stream from the natural gas reforming step passed straight to the PSA step and then to the bioreactor for fermentation. Alternatively, the gas stream from the natural gas reforming step may pass straight to the bioreactor for fermentation. These differing arrangements could be advantageous by reducing costs and any energy loss associated with the WGS step. Further, they may improve the fermentation process by providing a substrate having a higher CO content. The Water Gas Shift reaction is a know reaction having the following stoichiometry;

CO+H₂O→CO₂+H₂.

### The Reverse Water Gas Shift

The reverse water gas shift reaction (RWGS) is a method of producing carbon monoxide from hydrogen and carbon dioxide. In the presence of a suitable catalyst, the reaction takes place according to the following equation;

CO₂+H₂→CO+H₂O (ΔH=+9 kcal/mole)

Surprisingly we have found that we can use this reaction to make use of sources of hydrogen, particularly less desirable, impure streams containing hydrogen, with CO₂ to produce a CO containing gas substrate for feed to a bioreactor.

The RWGS reaction requires temperatures of approximately 400-600° C. The reaction requires a hydrogen-rich and/or a carbon dioxide-rich source. A CO₂ and/or H₂ source derived from a high temperature process such as gasification would be advantageous as it would alleviate the heat requirement for the reaction.

The RWGS reaction is an efficient method for CO₂ conversion as it requires a fraction of the power required for alternative CO₂ conversion methods such as solid-oxide or molten carbonate electrolysis.

Typically the RWGS reaction has been used to produce H₂O with CO as a by product. It has been of interest in the areas of space exploration, as when used in combination with a water electrolysis device, it would be capable of providing an oxygen source.

In accordance with the present invention, the RWGS reaction is used to produce CO, with H₂O being the by product. In industrial processes having H₂ and/or CO₂ waste gases, the RWGS reaction can be used to produce CO, which can then be used as a fermentation substrate in the bioreactor to produce one or more hydrocarbon product(s).

Ideal candidate streams for the reverse water gas shift reaction are low cost sources of H2 and/or CO2. Of particular interest are gas streams derived from a high temperature process such as a gasifier, as the reverse water gas shift reaction requires moderately high temperature conditions

According to one embodiment, the present invention provides a bioreactor which receives a CO and/or H₂ containing substrate from one or more of the previously described processes. The bioreactor contains a culture of one or more microorganisms capable of fermenting the CO and/or H₂ containing substrate to produce a hydrocarbon product. Thus, steps of a natural gas reforming process may be used to produce or improve the composition of a gaseous substrate for a fermentation process.

According to an alternative embodiment, at least one step of a natural gas reforming process may be improved by providing an output of a bioreactor to an element of a natural gas reforming process. Preferably, the output is a gas and may enhance efficiency and/or desired total product capture (for example of H₂) by the steam reforming process.

### Syngas Composition

There are a number of known methods for reforming a natural gas stream to produce syngas. The end use of the syngas can determine the optimal syngas properties. The type of reforming method, and the operating conditions used determines the syngas concentration. As such syngas composition depends on the choice of catalyst, reformer operating temperature and pressure, and the ratio of natural gas to CO₂, H₂O and/or O₂ or any combination of CO₂, H₂O and O₂. It would be understood to a person skilled in the art that a number of reforming technologies can be used to achieve a syngas with a desired composition.

Syngas compositions generated by various reforming technologies described above are generally in the range of;

Steam Methane Reforming: H₂/CO=3/1

Dry Reforming: H₂/CO=1/1

Partial Oxidation: H₂/CO=2/1

Auto-thermal reforming: H₂/CO=1.5/1 to 2.5/1 depending on the amount of steam and/or O₂ fed to the reformer.

These ranges relate to the syngas composition generated by the specific reforming reaction only; the actual syngas composition is determined by the extent of the main reforming reaction(s) in conjunction with various side reactions. The extent of such side reactions depends on the reactor temperature, pressure, feed-gas composition, and choice of catalyst. Such side reactions can include but are not limited to; water gas shift, reverse water gas shift, methane decomposition, the Boudouard reaction,

According to certain aspects of the invention the optimal H₂/CO ratio is between 1/1 and 2/1. Syngas streams having the desired composition range can be generated by a number of reforming options including, but not limited to; Steam methane reforming followed by Hydrogen removal; Partial oxidation followed by reverse water gas shift, auto-thermal reforming with the correct feed ratio of O₂ and/or H2O; or dry reforming with additional steam or O₂ in the reforming feed.

For desired syngas compositions of greater than 2:1 H₂/CO steam reforming is the favoured technology. Syngas compositions between 1/1 to 2/1 H₂/CO will generally require some form or combination of dry reforming, partial oxidation or auto-thermal reforming. Desired ratios of H₂/CO of <1 will generally require gas conditioning or gas separation in terms of hydrogen removal.

A skilled person would understand that these options are provided as an example of suitable methods and the invention is not limited to these particular combinations of technologies.

The syngas generated from natural gas reforming can be used as a feedstock for the microbial production of one or more products by fermentation. CO₂ may be produced as a by-product of an alcohol fermentation process wherein a syngas stream comprising CO and/or H₂ is fermented to produce ethanol. The CO₂ produced by the alcohol fermentation can be passed into a second bioreactor along with any unconverted H₂ to produce acetic acid in an acid fermentation reaction the acid fermentation reaction requires a gas stream having a H₂ and CO₂ composition of substantially 2:1. As would be understood by a skilled person, it is desirable to run the alcohol fermentation in such a way that the tail gas exiting the alcohol fermentation bioreactor has the desired composition for the acid fermentation reaction. In certain embodiments, the alcohol fermentation may be run in such a way that little or no H₂ is consumed during the fermentation. Table 1 shows the ratio of CO/H₂ required in the reformed natural gas stream entering the alcohol fermentation bioreactor to generate a tail-gas exiting the alcohol fermentation with a H₂:CO₂ ratio of 2:1.

In certain embodiments the H₂:CO₂ ratio of the tail gas is at least 1:1 or at least 2:1, or at least 3:1. In certain embodiments hydrogen and/or carbon dioxide is blended with the tail gas from the first bioreactor to provide a substrate having a H₂:CO₂ ratio of 2:1. In certain embodiments at least a portion of H₂ or CO₂ is removed from the tail gas exiting the first bioreactor to provide a substrate having a H₂:CO₂ ratio of substantially 2:1.

CO₂ may be a by-product of several reforming reactions. If the alcohol fermentation consumes a large portion of hydrogen then it may be difficult to achieve the desired H₂:CO₂ ratio in the tail gas exiting the alcohol fermentation, without the use of additional hydrogen. In certain embodiments it may be desirable to separate at least a portion of the hydrogen from the syngas stream, prior to the syngas stream being passed into the alcohol fermentation. The separated H₂ may then be blended with the tail gas exiting the alcohol fermentation

### Fermentation

### The Bioreactor

The fermentation may be carried out in any suitable bioreactor, such as a continuous stirred tank reactor (CSTR), an immobilised cell reactor, a gas-lift reactor, a bubble column reactor (BCR), a membrane reactor, such as a Hollow Fibre Membrane Bioreactor (HFMBR) or a trickle bed reactor (TBR). Also, in some embodiments of the invention, the bioreactor may comprise a first, growth reactor in which the micro-organisms are cultured, and a second, fermentation reactor, to which fermentation broth from the growth reactor may be fed and in which most of the fermentation product (e.g. ethanol and acetate) may be produced. The bioreactor of the present invention is adapted to receive a CO and/or H₂ containing substrate.

The CO and/or H₂ Containing Substrate

The CO and/or H₂ containing substrate is captured or channeled from the process using any convenient method. Depending on the composition of the CO and/or H₂ containing substrate, it may also be desirable to treat it to remove any undesired impurities, such as dust particles before introducing it to the fermentation. For example, the substrate may be filtered or scrubbed using known methods.

The substrate comprising CO, preferably a gaseous substrate may be obtained as a by-product of a natural gas reforming process. Such natural gas reforming reactions include steam methane reforming, partial oxidation, dry reforming, auto-thermal reforming, water gas shift reactions, reverse water gas shift reactions, as well as coking reactions such as methane decomposition or the Boudouard reaction.

Typically, the CO will be added to the fermentation reaction in a gaseous state. However, methods of the invention are not limited to addition of the substrate in this state. For example, the carbon monoxide can be provided in a liquid. For example, a liquid may be saturated with a carbon monoxide containing gas and that liquid added to the bioreactor. This may be achieved using standard methodology. By way of example a microbubble dispersion generator (Hensirisak et. al. Scale-up of microbubble dispersion generator for aerobic fermentation; Applied Biochemistry and Biotechnology Volume 101 Number 3/October, 2002) could be used for this purpose. Where a “gas stream” is referred to herein, the term also encompasses other forms of transporting the gaseous components of that stream such as the saturated liquid method described above.

### Gas Compositions

The CO-containing substrate may contain any proportion of CO, such as at least about 20% to about 100% CO by volume, from 40% to 95% CO by volume, from 40% to 60% CO by volume, and from 45% to 55% CO by volume. In particular embodiments, the substrate comprises about 25%, or about 30%, or about 35%, or about 40%, or about 45%, or about 50% CO, or about 55% CO, or about 60% CO by volume. Substrates having lower concentrations of CO, such as 2%, may also be appropriate, particularly when H₂ and CO₂ are also present.

The presence of H₂ should not be detrimental to hydrocarbon product formation by fermentation. In particular embodiments, the presence of hydrogen results in an improved overall efficiency of alcohol production. For example, in particular embodiments, the substrate may comprise an approximate 2:1, or 1:1, or 1:2 ratio of H₂:CO. In other embodiments, the CO containing substrate comprises less than about 30% H₂, or less than 27% H₂, or less than 20% H₂, or less than 10% H₂, or lower concentrations of H₂, for example, less than 5%, or less than 4%, or less than 3%, or less than 2%, or less than 1%, or is substantially hydrogen free. In still other embodiments, the CO containing substrate comprises greater than 50% H₂, or greater than 60% H₂, or greater than 70% H₂, or greater than 80% H₂, or greater than 90% H₂.

According to some embodiments of the invention a Pressure Swing Adsorption (PSA) step recovers hydrogen from the substrate received from the SR or WGS steps. In a typical embodiment, the substrate exiting the PSA step comprises about 10-35% H₂. The H₂ may pass through the bioreactor and be recovered from the substrate. In a particular embodiment of the invention, the H₂ is recycled to the PSA to be recovered from the substrate.

The substrate may also contain some CO₂ for example, such as about 1% to about 80% CO₂ by volume, or 1% to about 30% CO₂ by volume.

### Fermentation

Processes for the production of ethanol and other alcohols from gaseous substrates are known. Exemplary processes include those described for example in WO2007/117157, WO2008/115080, WO2009/022925, WO2009/064200, U.S. Pat. No. 6,340,581, U.S. Pat. No. 6,136,577, U.S. Pat. No. 5,593,886, U.S. Pat. No. 5,807,722 and U.S. Pat. No. 5,821,111, each of which is incorporated herein by reference.

### Microorganisms

In various embodiments, the fermentation is carried out using a culture of one or more strains of carboxydotrophic bacteria. In various embodiments, the carboxydotrophic bacterium is selected from Moorella, Clostridium, Ruminococcus, Acetobacterium, Eubacterium, Butyribacterium, Oxobacter, Methanosarcina, Methanosarcina, and Desulfotomaculum. A number of anaerobic bacteria are known to be capable of carrying out the fermentation of CO to alcohols, including n-butanol and ethanol, and acetic acid, and are suitable for use in the process of the present invention.

In a further embodiment, the microorganism is selected from a cluster of carboxydotrophic Clostridia comprising the species C. autoethanogenum, C. ljungdahlii, and “C. ragsdalei” and related isolates.

The strains of this cluster are defined by common characteristics, having both a similar genotype and phenotype, and they all share the same mode of energy conservation and fermentative metabolism. The strains of this cluster lack cytochromes and conserve energy via an Rnf complex.

All strains of this cluster have a similar genotype with a genome size of around 4.2 MBp (Köpke et al., 2010) and a GC composition of around 32% mol (Abrini et al., 1994; Köpke et al., 2010; Tanner et al., 1993) (WO 2008/028055; US patent 2011/0229947), and conserved essential key gene operons encoding for enzymes of Wood-Ljungdahl pathway (Carbon monoxide dehydrogenase, Formyl-tetrahydrofolate synthetase, Methylene-tetrahydrofolate dehydrogenase, Formyl-tetrahydrofolate cyclohydrolase, Methylene-tetrahydrofolate reductase, and Carbon monoxide dehydrogenase/Acetyl-CoA synthase), hydrogenase, formate dehydrogenase, Rnf complex (rnfCDGEAB), pyruvate:ferredoxin oxidoreductase, aldehyde:ferredoxin oxidoreductase (Köpke et al., 2010, 2011). The organization and number of Wood-Ljungdahl pathway genes, responsible for gas uptake, has been found to be the same in all species, despite differences in nucleic and amino acid sequences (Köpke et al., 2011).

The strains all have a similar morphology and size (logarithmic growing cells are between 0.5-0.7×3-5 μm), are mesophilic (optimal growth temperature between 30-37° C.) and strictly anaerobe (Abrini et al., 1994; Tanner et al., 1993)(WO 2008/028055). Moreover, they all share the same major phylogenetic traits, such as same pH range (pH 4-7.5, with an optimal initial pH of 5.5-6), strong autotrophic growth on CO containing gases with similar growth rates, and a similar metabolic profile with ethanol and acetic acid as main fermentation end product, and small amounts of 2,3-butanediol and lactic acid formed under certain conditions (Abrini et al., 1994; Köpke et al., 2011; Tanner et al., 1993)(WO 2008/028055). Indole production was observed with all species. However, the species differentiate in substrate utilization of various sugars (e.g. rhamnose, arabinose), acids (e.g. gluconate, citrate), amino acids (e.g. arginine, histidine), or other substrates (e.g. betaine, butanol). Moreover some of the species were found to be auxotroph to certain vitamins (e.g. thiamine, biotin) while others were not. These traits are therefore not specific to one organism like C. autoethanogenum or C. ljungdahlii, but rather general traits for carboxydotrophic, ethanol-synthesizing Clostridia and it can be anticipated that mechanism work similar across these strains, although there may be differences in performance. Examples of such bacteria that are suitable for use in the invention include those of the genus Clostridium, such as strains of Clostridium ljungdahlii, including those described in WO 00/68407, EP 117309, U.S. Pat. Nos. 5,173,429, 5,593,886, and 6,368,819, WO 98/00558 and WO 02/08438, Clostridium carboxydivorans (Liou et al., International Journal of Systematic and Evolutionary Microbiology 33: pp 2085-2091), Clostridium ragsdalei (WO/2008/028055) and Clostridium autoethanogenum (Abrini et al, Archives of Microbiology 161: pp 345-351). Other suitable bacteria include those of the genus Moorella, including Moorella sp HUC22-1, (Sakai et al, Biotechnology Letters 29: pp 1607-1612), and those of the genus Carboxydothermus (Svetlichny, V. A., Sokolova, T. G. et al (1991), Systematic and Applied Microbiology 14: 254-260). Further examples include Moorella thermoacetica, Moorella thermoautotrophica, Ruminococcus productus, Acetobacterium woodii, Eubacterium limosum, Butyribacterium methylotrophicum, Oxobacter pfennigii, Methanosarcina barkeri, Methanosarcina acetivorans, Desulfotomaculum kuznetsovii (Simpa et. al. Critical Reviews in Biotechnology, 2006 Vol. 26. Pp41-65). In addition, it should be understood that other acetogenic anaerobic bacteria may be applicable to the present invention as would be understood by a person of skill in the art. It will also be appreciated that the invention may be applied to a mixed culture of two or more bacteria.

One exemplary micro-organism suitable for use in the present invention is Clostridium autoethanogenum. In one embodiment, the Clostridium autoethanogenum is a Clostridium autoethanogenum having the identifying characteristics of the strain deposited at the German Resource Centre for Biological Material (DSMZ) under the identifying deposit number 19630. In other embodiments, the Clostridium autoethanogenum is a Clostridium autoethanogenum having the identifying characteristics of DSMZ deposit number DSMZ 10061 or DSMZ deposit number DSMZ 23693. These strains have a particular tolerance to changes in substrate composition, particularly of H₂ and CO and as such are particularly well suited for use in combination with a natural gas reforming process.

Culturing of the bacteria used in the methods of the invention may be conducted using any number of processes known in the art for culturing and fermenting substrates using anaerobic bacteria. By way of example, those processes generally described in the following articles using gaseous substrates for fermentation may be utilised: (i) K. T. Klasson, et al. (1991). Bioreactors for synthesis gas fermentations resources. Conservation and Recycling, 5; 145-165; (ii) K. T. Klasson, et al. (1991). Bioreactor design for synthesis gas fermentations. Fuel. 70. 605-614; (iii) K. T. Klasson, et al. (1992). Bioconversion of synthesis gas into liquid or gaseous fuels. Enzyme and Microbial Technology. 14; 602-608; (iv) J. L. Vega, et al. (1989). Study of Gaseous Substrate Fermentation: Carbon Monoxide Conversion to Acetate. 2. Continuous Culture. Biotech. Bioeng. 34. 6. 785-793; (v) J. L. Vega, et al. (1989). Study of gaseous substrate fermentations: Carbon monoxide conversion to acetate. 1. Batch culture. Biotechnology and Bioengineering. 34. 6. 774-784; (vi) J. L. Vega, et al. (1990). Design of Bioreactors for Coal Synthesis Gas Fermentations. Resources, Conservation and Recycling. 3. 149-160; all of which are incorporated herein by reference.

### Fermentation Conditions

It will be appreciated that for growth of the bacteria and CO-to-hydrocarbon fermentation to occur, in addition to the CO-containing substrate, a suitable liquid nutrient medium will need to be fed to the bioreactor. A nutrient medium will contain vitamins and minerals sufficient to permit growth of the micro-organism used. Anaerobic media suitable for the production of hydrocarbon products through fermentation using CO as the sole carbon source are known in the art. For example, suitable media are described in U.S. Pat. Nos. 5,173,429 and 5,593,886 and WO 02/08438, WO2007/115157 and WO2008/115080 referred to above.

The fermentation should desirably be carried out under appropriate conditions for the desired fermentation to occur (e.g. CO-to-ethanol). Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO in the liquid phase does not become limiting, and maximum product concentrations to avoid product inhibition. Suitable conditions are described in WO02/08438, WO07/117157 and WO08/115080.

The optimum reaction conditions will depend partly on the particular micro-organism used. However, in general, it is preferred that the fermentation be performed at pressure higher than ambient pressure. Operating at increased pressures allows a significant increase in the rate of CO transfer from the gas phase to the liquid phase where it can be taken up by the micro-organism as a carbon source for the production of hydrocarbon products. This in turn means that the retention time (defined as the liquid volume in the bioreactor divided by the input gas flow rate) can be reduced when bioreactors are maintained at elevated pressure rather than atmospheric pressure. Also, since a given CO-to-hydrocarbon conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment. According to examples given in U.S. Pat. No. 5,593,886, reactor volume can be reduced in linear proportion to increases in reactor operating pressure, i.e. bioreactors operated at 10 atmospheres of pressure need only be one tenth the volume of those operated at 1 atmosphere of pressure.

The benefits of conducting a gas-to-hydrocarbon fermentation at elevated pressures have also been described elsewhere. For example, WO 02/08438 describes gas-to-ethanol fermentations performed under pressures of 2.1 atm and 5.3 atm, giving ethanol productivities of 150 g/l/day and 369 g/l/day respectively. However, example fermentations performed using similar media and input gas compositions at atmospheric pressure were found to produce between 10 and 20 times less ethanol per litre per day.

It is also desirable that the rate of introduction of the CO-containing gaseous substrate is such as to ensure that the concentration of CO in the liquid phase does not become limiting. This is because a consequence of CO-limited conditions may be that the hydrocarbon product is consumed by the culture.

### Fermentation Products

Methods of the invention can be used to produce any of a variety of hydrocarbon products. This includes alcohols, acids and/or diols. More particularly, the invention may be applicable to fermentation to produce butyrate, propionate, caproate, ethanol, propanol, butanol, 2,3-butanediol, propylene, butadiene, iso-butylene, and ethylene. These and other products may be of value for a host of other processes such as the production of plastics, pharmaceuticals and agrochemicals. In a particular embodiment, the fermentation product is used to produce gasoline range hydrocarbons (about 8 carbon), diesel hydrocarbons (about 12 carbon) or jet fuel hydrocarbons (about 12 carbon).

In certain embodiments of the invention, at least a portion of CO₂ produced as a by-product of the alcohol fermentation process is reused in the reforming process. In certain embodiments, CO₂ produced in the alcohol fermentation process is passed to a reforming process such as dry reforming, wherein the CO₂ is reacted with methane to produce syngas. In another embodiment, CO₂ produced in a fermentation process is passed to a Partial Oxidation Reforming module, where it is reacted with methane to produce syngas, In a further embodiment CO₂ produced in a fermentation process is passed to an Autothermal Reforming module, wherein the CO₂ is reacted with methane to produce syngas.

The invention also provides that at least a portion of a hydrocarbon product produced by the fermentation is reused in the natural gas reforming process. This may be performed because hydrocarbons other than CH₄ are able to react with steam over a catalyst to produce H₂ and CO. In a particular embodiment, ethanol is recycled to be used as a feedstock for the steam reforming process. In a further embodiment, the hydrocarbon feedstock and/or product is passed through a prereformer prior to being used in the reforming process. Passing through a prereformer partially completes the reforming step of the reforming process which can increase the efficiency of natural gas conversion to syngas and reduce the required capacity of the reforming furnace.

The methods of the invention can also be applied to aerobic fermentations, and to anaerobic or aerobic fermentations of other products, including but not limited to isopropanol.

### Product Recovery

The products of the fermentation reaction can be recovered using known methods. Exemplary methods include those described in WO07/117157, WO08/115080, U.S. Pat. No. 6,340,581, U.S. Pat. No. 6,136,577, U.S. Pat. No. 5,593,886, U.S. Pat. No. 5,807,722 and U.S. Pat. No. 5,821,111. However, briefly and by way of example ethanol may be recovered from the fermentation broth by methods such as fractional distillation or evaporation, and extractive fermentation.

Distillation of ethanol from a fermentation broth yields an azeotropic mixture of ethanol and water (i.e., 95% ethanol and 5% water). Anhydrous ethanol can subsequently be obtained through the use of molecular sieve ethanol dehydration technology, which is also well known in the art.

Extractive fermentation procedures involve the use of a water-miscible solvent that presents a low toxicity risk to the fermentation organism, to recover the ethanol from the dilute fermentation broth. For example, oleyl alcohol is a solvent that may be used in this type of extraction process. Oleyl alcohol is continuously introduced into a fermenter, whereupon this solvent rises forming a layer at the top of the fermenter which is continuously extracted and fed through a centrifuge. Water and cells are then readily separated from the oleyl alcohol and returned to the fermenter while the ethanol-laden solvent is fed into a flash vaporization unit. Most of the ethanol is vaporized and condensed while the oleyl alcohol is non volatile and is recovered for re-use in the fermentation.

Acetate, which may be produced as a by-product in the fermentation reaction, may also be recovered from the fermentation broth using methods known in the art.

For example, an adsorption system involving an activated charcoal filter may be used. In this case, it is preferred that microbial cells are first removed from the fermentation broth using a suitable separation unit. Numerous filtration-based methods of generating a cell free fermentation broth for product recovery are known in the art. The cell free ethanol—and acetate—containing permeate is then passed through a column containing activated charcoal to adsorb the acetate. Acetate in the acid form (acetic acid) rather than the salt (acetate) form is more readily adsorbed by activated charcoal. It is therefore preferred that the pH of the fermentation broth is reduced to less than about 3 before it is passed through the activated charcoal column, to convert the majority of the acetate to the acetic acid form.

Acetic acid adsorbed to the activated charcoal may be recovered by elution using methods known in the art. For example, ethanol may be used to elute the bound acetate. In certain embodiments, ethanol produced by the fermentation process itself may be used to elute the acetate. Because the boiling point of ethanol is 78.8° C. and that of acetic acid is 107° C., ethanol and acetate can readily be separated from each other using a volatility-based method such as distillation.

Other methods for recovering acetate from a fermentation broth are also known in the art and may be used. For example, U.S. Pat. Nos. 6,368,819 and 6,753,170 describe a solvent and cosolvent system that can be used for extraction of acetic acid from fermentation broths. As with the example of the oleyl alcohol-based system described for the extractive fermentation of ethanol, the systems described in U.S. Pat. Nos. 6,368,819 and 6,753,170 describe a water immiscible solvent/co-solvent that can be mixed with the fermentation broth in either the presence or absence of the fermented micro-organisms in order to extract the acetic acid product. The solvent/co-solvent containing the acetic acid product is then separated from the broth by distillation. A second distillation step may then be used to purify the acetic acid from the solvent/co-solvent system.

The products of the fermentation reaction (for example ethanol and acetate) may be recovered from the fermentation broth by continuously removing a portion of the broth from the fermentation bioreactor, separating microbial cells from the broth (conveniently by filtration), and recovering one or more product from the broth simultaneously or sequentially. In the case of ethanol it may be conveniently recovered by distillation, and acetate may be recovered by adsorption on activated charcoal, using the methods described above. The separated microbial cells are preferably returned to the fermentation bioreactor. The cell free permeate remaining after the ethanol and acetate have been removed is also preferably returned to the fermentation bioreactor. Additional nutrients (such as B vitamins) may be added to the cell free permeate to replenish the nutrient medium before it is returned to the bioreactor. Also, if the pH of the broth was adjusted as described above to enhance adsorption of acetic acid to the activated charcoal, the pH should be re-adjusted to a similar pH to that of the broth in the fermentation bioreactor, before being returned to the bioreactor.

Biomass recovered from the bioreactor may undergo anaerobic digestion in a digestion to produce a biomass product, preferably methane. This biomass product may be used as a feedstock for the steam reforming process or used to produce supplemental heat to drive one or more of the reactions defined herein.

### Gas Separation/Production

The fermentation of the present invention has the advantage that it is robust to the use of substrates with impurities and differing gas concentrations. Accordingly, production of a hydrocarbon product still occurs when a wide range of gas compositions is used as a fermentation substrate. The fermentation reaction may also be used as a method to separate and/or capture particular gases (for example CO) from the substrate and to concentrate gases, for example H₂, for subsequent recovery. When used in conjunction with one or more other steps of a natural gas reforming process as defined herein, the fermentation reaction may reduce the concentration of CO in the substrate and consequently concentrate H₂ which enables improved H₂ recovery.

The gas separation module is adapted to receive a gaseous substrate from the bioreactor and to separate one or more gases from one or more other gases. The gas separation may comprise a PSA module, preferably adapted to recover hydrogen from the substrate. In a particular embodiment, the gaseous substrate from the natural gas reforming process is fed directly to the bioreactor, then the resulting post-fermentation substrate passed to a gas separation module. This preferred arrangement has the advantage that gas separation is easier due to the removal of one or more impurities from the stream. The impurity may be CO. Additionally, this preferred arrangement would convert some gases to more easily separated gases, for example CO would be converted to CO₂.

### CO₂ and H₂ Fermentation

A number of anaerobic bacteria are known to be capable of carrying out the fermentation of CO₂ and H₂ to alcohols, including ethanol, and acetic acid, and are suitable for use in the process of the present invention. Acetogens have the ability to convert gaseous substrates such as H₂, CO₂ and CO into products including acetic acid, ethanol and other fermentation products by the Wood-Ljungdahl pathway. Examples of such bacteria that are suitable for use in the invention include those of the genus Acetobacterium, such as strains of Acetobacterium woodii ((Demler, M., Weuster-Botz, “Reaction Engineering Analysis of Hydrogenotrophic Production of Acetic Acid by Acetobacterum Woodii”, Biotechnology and Bioengineering, Vol. 108, No. 2, February 2011) and.

Acetobacterium woodii has been shown to produce acetate by fermentation of gaseous substrates comprising CO₂ and H₂. Buschhorn et al. demonstrated the ability of A. woodii to produce ethanol in a glucose fermentation with a phosphate limitation.

Other suitable bacteria include those of the genus Moorella, including Moorella sp HUC22-1, (Sakai et al, Biotechnology Letters 29: pp 1607-1612), and those of the genus Carboxydothermus (Svetlichny, V. A., Sokolova, T. G. et al (1991), Systematic and Applied Microbiology 14: 254-260). Further examples include Moorella thermoacetica, Moorella thermoautotrophica, Ruminococcus productus, Acetobacterium woodii, Eubacterium limosum, Butyribacterium methylotrophicum, Oxobacter pfennigii, Methanosarcina barkeri, Methanosarcina acetivorans, Desulfotomaculum kuznetsovii (Simpa et. al. Critical Reviews in Biotechnology, 2006 Vol. 26. Pp41-65). In addition, it should be understood that other acetogenic anaerobic bacteria may be applicable to the present invention as would be understood by a person of skill in the art. It will also be appreciated that the invention may be applied to a mixed culture of two or more bacteria.

One exemplary micro-organism suitable for use in the present invention is Acetobacterium woodii having the identifying characteristics of the strain deposited at the German Resource Centre for Biological Material (DSMZ) under the identifying deposit number DSM 1030.

### The CO₂ and H₂ Containing Substrate

Preferably the carbon source for the fermentation can be a gaseous substrate comprising carbon dioxide in combination with hydrogen. Similarly, the gaseous substrate may be a CO₂ and H₂ containing waste gas obtained as a by-product of an industrial process, or from some other source. The largest source of CO₂ emissions globally is from the combustion of fossil fuels such as coal, oil and gas in power plants, industrial facilities and other sources.

The gaseous substrate may be a CO₂ and H₂-containing waste gas obtained as a by-product of an industrial process, or from some another source such as from automobile exhaust fumes. In certain embodiments, the industrial process is selected from the group consisting of hydrogen manufacture, ammonia manufacture, combustion of fuels, gasification of coal, and the production of limestone and cement. The gaseous substrate may be the result of blending one or more gaseous substrates to provide a blended stream. It would be understood to a skilled person that waste gas streams rich in H₂ or rich in CO₂ are more abundant than waste gas streams rich in both H₂ and CO₂. A skilled person would understand that blending one or more gas streams comprising one of the desired components of CO₂ and H₂ would fall within the scope of the present invention. In preferred embodiments the ratio of H₂:CO₂ in the substrate is 2:1.

Hydrogen rich gas streams are produced by a variety of processes including reformation of hydrocarbons, and in particular reformation of natural gas. Other sources of hydrogen rich gas include the electrolysis of water, by-products from electrolytic cells used to produce chlorine and from various refinery and chemical streams.

Gas streams typically rich in Carbon dioxide include exhaust gasses from combustion of a hydrocarbon, such as natural gas or oil. Carbon dioxide is also produced as a by-product from the production of ammonia, lime or phosphate and from natural carbon dioxide wells.

### Carbon Capture

Certain natural gas reforming processes produce a substantial quantity of CO₂ which is emitted to the atmosphere. However, CO₂ is a greenhouse gas that contributes to climate change. There is considerable pressure on industry to reduce carbon (including CO₂) emissions and efforts are underway to capture the carbon prior to emission. Economic incentives for reducing carbon emissions and emissions trading schemes have been established in several jurisdictions in an effort to incentivise industry to limit carbon emissions.

The present invention captures carbon from a substrate containing CO and/or H₂ and/or CO₂ and/or CH₄ via a fermentation process and produces a valuable hydrocarbon product (“valuable” is interpreted as being potentially useful for some purpose and not necessarily a monetary value). In the absence of the fermentation of the present invention, the CO and CH₄ would be likely to be burned to release energy and the resulting CO₂ emitted to the atmosphere. Where the energy produced is used to generate electricity, there are likely to be considerable losses in energy due to the transmission along high-voltage power lines. In contrast, the hydrocarbon product produced by the present invention may be easily transported and delivered in a usable form to industrial, commercial, residential and transportation end-users resulting in increased energy efficiency and convenience. The production of hydrocarbon products that are formed from what are effectively waste gases is an attractive proposition for industry. This is especially true for industries situated in remote locations if it is logistically feasible to transport the product long distances.

The WGS step produces CO₂ as a by-product. In certain aspects of the invention the omission of the WGS step and passing of the reformed gas stream straight to the PSA or bioreactor, reduces the amount of CO₂ available. Where the CO in the fermentation substrate is converted to a hydrocarbon product such as ethanol, this reduces or eliminates the emission of CO₂ to the atmosphere by the industrial plant.

Alternatively, the CO₂ may be recycled to the bioreactor, preferably in combination with a substrate comprising H₂. As noted hereinbefore, fermentations used in embodiments of the invention may use substrates containing H₂ and CO₂.

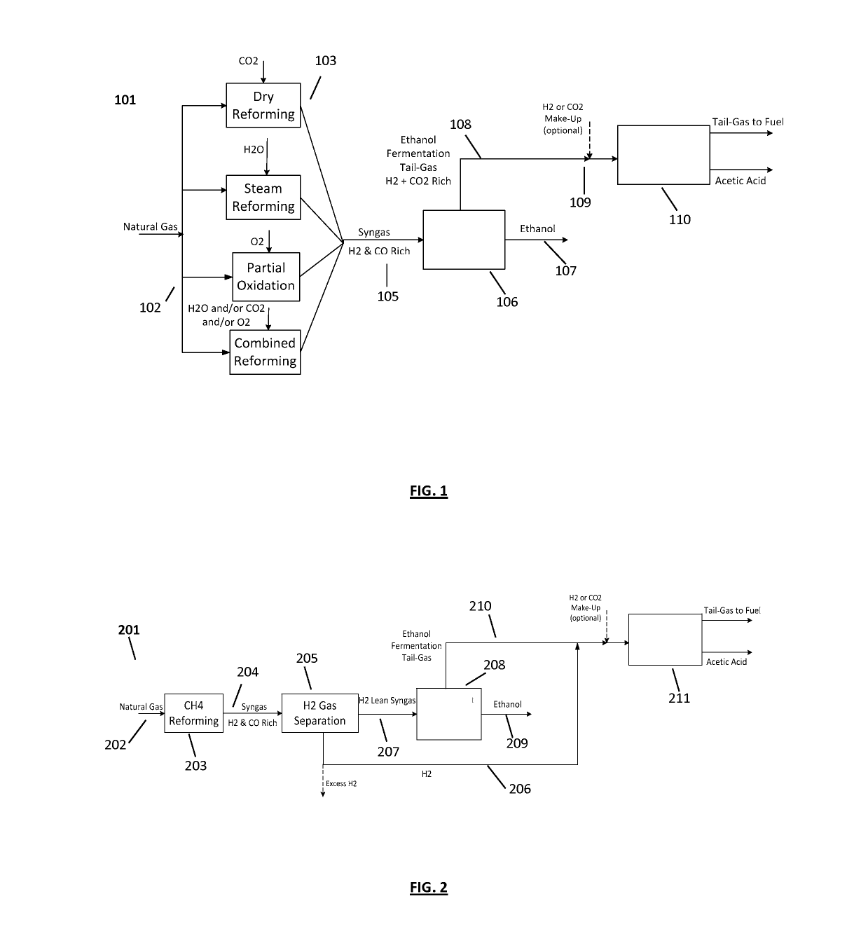

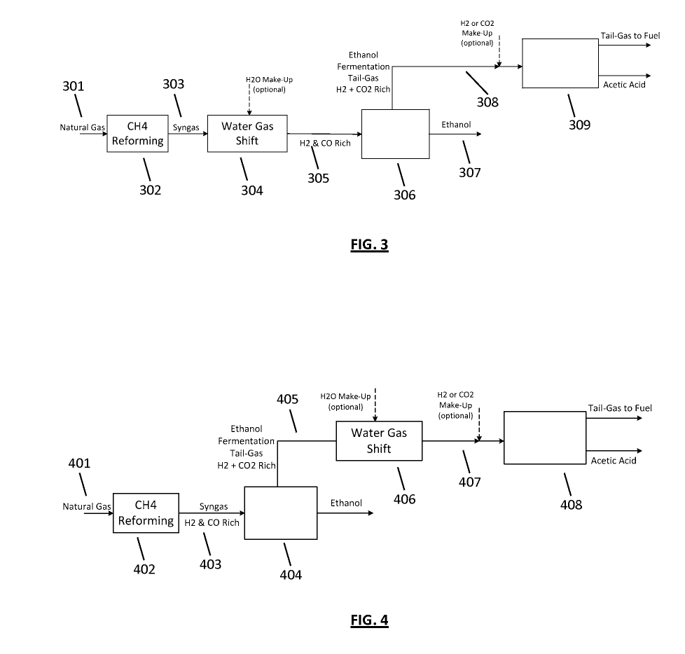

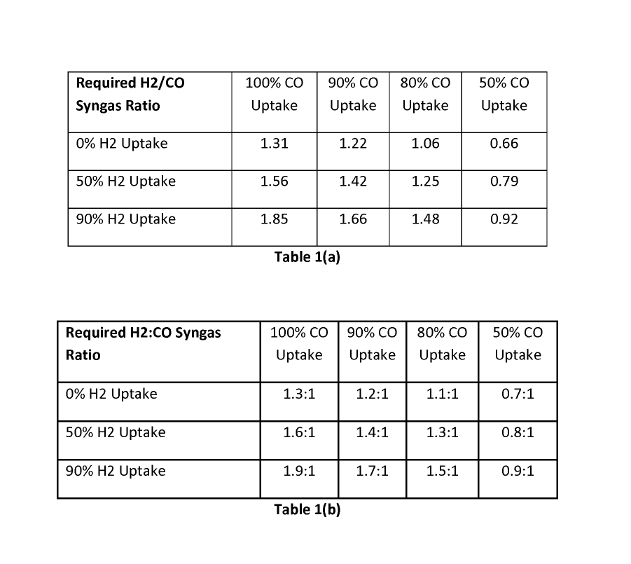

Various embodiments of systems of the invention are described in the accompanying Figures. Descriptions of certain aspects of embodiments are the same in FIGS. 2 and 3 as they are in FIG. 1. Descriptions of said aspects will not be repeated (i.e. A first bioreactor is described in FIG. 1 and the first bioreactor of FIG. 2 has the same feature, therefor no further definition is given of the first bioreactor in FIG. 2).

FIG. 1 is a schematic representation of a system 101 according to one embodiment of the invention. A gas stream comprising methane enters the system 101 via a suitable conduit 102. The natural gas substrate stream comprises at least methane (CH₄). The conduit 102 delivers the natural gas stream to a reforming stage 103 where the natural gas is converted to a syngas stream comprising at least CO, H₂ and CO₂. The reforming stage 103 comprises at least one module selected from the group comprising; a dry reforming module; a steam reforming module; a partial oxidation module; and a combined reforming module, The syngas exits the reforming stage 103 via a syngas conduit 104 and is flowed to a first bioreactor 106 for use as a syngas substrate. The syngas entering the first bioreactor has a H₂:CO ratio of at least 1:2 or at least 1:1 or at least 2:1 or at least 3:1 or at least 4:1 or at least 5:1.

The bioreactor 106 comprises a liquid nutrient medium comprising a culture of Clostridium autoethanogenum. The culture ferments the syngas substrate to produce one or more alcohols and a tail gas comprising CO₂ and H₂. The uptake of CO and H₂ by the culture is controlled such that the tail gas comprising CO2 and H2 has a desired composition. For example the CO₂ and H₂ tail gas can comprise H₂ and CO₂ at a ratio of 1:1 or 2:1 or 3:1. The desired tail gas composition is H₂:CO₂ at a ratio of 2:1. The ratio of CO and H₂ in the syngas substrate can be adjusted to enable a tail gas having the desired H₂:CO₂ ratio. Table 1 shows the CO:H₂ ratios required in the syngas depending on the uptake of CO and H₂ by the culture, to provide a tail gas having a H₂:CO₂ ratio of 2:1.

The one or more alcohols exits the first bioreactor 106 in a fermentation broth stream via a conduit 107. The one or more alcohols are recovered from the fermentation broth stream by known methods such as distillation, evaporation, and extractive fermentation.

The tail gas comprising H₂ and CO₂ exits the first bioreactor via a conduit 108 and is flowed to a second bioreactor 110. Optionally additional H₂ and/or CO₂ is blended with tail gas to provide a H₂ and CO₂ stream having a ratio of 2:1. The second bioreactor 110 comprises a liquid nutrient medium comprising a culture of Acetobacterium woodii. The culture ferments the H₂:CO₂ substrate to produce acetic acid according to the following stoichiometric equation 4H₂+2CO₂→CH₃COOH+2H₂O.

FIG. 2 is a schematic representation of a system according to a second embodiment of the invention. According to FIG. 2, a gas stream comprising methane is flowed into a methane reforming module 203 via a conduit 202. The natural gas stream is reformed to produce a syngas stream comprising at least CO, CO₂ and H₂. The syngas stream exits the methane reforming module via a conduit 204 and is flowed to a Hydrogen separation module 205, wherein at least a portion of the hydrogen is separated from the syngas stream to provide a hydrogen depleted syngas stream. The separated hydrogen exits the hydrogen separation module 205 via a conduit 206. The hydrogen depleted syngas stream exits the hydrogen separation module via a conduit 207 and flowed into a first bioreactor 208. The hydrogen depleted syngas stream is fermented in the first bioreactor 208 to produce ethanol and a tail gas stream comprising CO₂ and H₂. As for FIG. 1, the composition of the tail gas comprising H₂ and CO₂ is dependent on the composition of the substrate entering the bioreactor and the amount of CO and H₂ consumed (uptake) by the culture. The preferred ratio of H₂ and CO₂ in the tail gas exiting the bioreactor is 2:1.

The tail gas comprising H₂ and CO₂ exits the bioreactor via a conduit 210 and is flowed to a second bioreactor 211. If the H₂:CO₂ ratio of the tail gas is not 2:1 additional Hydrogen and/or CO₂ can be blended with the tail gas before it enters the second bioreactor. If required a portion of the separated hydrogen can be supplied to tail gas via the conduit 207. Excess hydrogen can be used for fuel or energy or other known applications.

The culture in the second bioreactor 211 ferments the H₂ and CO₂ to produce acetic acid. The acetic acid is recovered by known methods.

FIG. 3A is a schematic representation of a system according to another embodiment of the invention. In FIG. 3A a gas stream comprising methane is passed to a methane reforming module 302 where it is converted to a syngas substrate. In this embodiment the syngas produced by the reforming module 302 is rich in CO. The CO-rich syngas substrate is flowed from the methane reforming module 302 to a Water Gas Shift module 304 via a conduit 303. At least a portion of the CO is converted to CO₂ and H₂ in the water gas shift module. The hydrogen rich gas stream exiting the Water Gas Shift module 304 is passed, via a conduit 305, to a first bioreactor 306 wherein at least a portion of the CO and optionally H₂ are fermented to produce ethanol and a H₂/CO₂ tail gas. The ethanol produced in the first bioreactor is recovered by know methods. The H₂ and CO₂ tail gas is flowed from the first bioreactor 302 via a conduit 308 to a second bioreactor 309. As for FIG. 2, if the tail gas does not have the desired H₂:CO₂ ratio, additional H₂ and/or CO₂ can be blended with the tail gas. The H₂/CO₂ substrate is fermented in the first bioreactor to produce acetic acid. The acetic acid produced by the first bioreactor is recovered by known methods.

FIG. 4 is a schematic representation of a system according to another embodiment of the invention. In FIG. 4, the gas stream comprising methane is provided to a methane reforming module 402 and produces a syngas rich in CO and H₂. The CO and H₂ rich syngas is flowed from the methane reforming module 402, via a conduit 403, to a first bioreactor 404, where at least a portion of the CO and optionally H2 is fermented to produce ethanol and a tail gas comprising CO₂ and H₂. The tail gas comprising CO₂ and H₂ is passed via a conduit 405 to a water gas shift module 406 wherein any CO remaining in the tail gas in converted to CO₂ and H₂ to provide an exit gas rich in CO₂ and H₂. The exit gas is passed via a conduit 407 to a second bioreactor 408. Additional CO₂ and/or H₂ is blended with the exit stream to provide a stream having a 2:1 H₂ to CO₂ ratio to the bioreactor. The H₂ and CO₂ is fermented in the bioreactor to produce acetic acid.

In any of the above Figures, a tail gas exiting the bioreactor can be passed back to the reforming module.

The reference to any prior art in this specification is not, and should not be taken as, an acknowledgement or any form of suggestion that that prior art forms part of the common general knowledge in the field of endeavour in any country.

Throughout this specification and any claims which follow, unless the context requires otherwise, the words “comprise”, “comprising” and the like, are to be construed in an inclusive sense as opposed to an exclusive sense, that is to say, in the sense of “including, but not limited to”.

What we claim is:

1. A method for producing at least one alcohol and at least one acid from a gas stream comprising methane, the method comprising;
a) Flowing the gas stream to a reforming module and reforming the gas stream to produce a syngas substrate comprising CO, CO₂ and H₂;
b) Flowing the syngas substrate to a first bioreactor, the first bioreactor comprising a liquid nutrient media comprising a culture of one or more carboxydotrophic micro-organisms;
c) Fermenting the syngas substrate to produce at least one alcohol and a tail gas stream comprising H₂ and CO₂;
d) Flowing the tail gas stream to a second bioreactor, the second bioreactor comprising a liquid nutrient medium comprising a culture of one or more microorganism; and
e) Fermenting the tail gas stream to produce one or more acids;
wherein the composition of the tail gas stream exiting the first bioreactor is controlled at a desired ratio of H₂:CO₂ by measuring the amount of CO and H₂ consumed by the one or more carboxydotrophic microorganism and adjusting the syngas substrate in response to changes in the amount of CO and H₂ consumed.

2. The method of claim 1 wherein the reforming module is selected from the group comprising: dry reforming, steam reforming, partial oxidation and auto thermal reforming.

3. The method of claim 1 wherein the syngas substrate provided to the first bioreactor comprise CO, CO₂ and H₂ at a composition such that the tail gas stream exiting the first bioreactor comprises H₂ and CO₂ at a ratio of between 1:2 and 3:1.

4. The method of claim 3 wherein additional H₂ and/or CO₂ is added to the tail gas exiting the first bioreactor to provide a H₂ and CO₂ substrate having a H₂:CO₂ ratio of 2:1.

5. The method of claim 1 wherein the syngas substrate provided to the first bioreactor comprises H₂ and CO at a ratio of between 0.5:1 and 5:1.

6. The method of claim 5 wherein the syngas substrate provided to the first bioreactor comprises H₂ and CO at a ratio of 0.7:1 to 1.9:1.

7. The method of claim 1 where the gas stream is a natural gas stream.

8. The method of claim 1 wherein CO₂ and/or H₂ is blended with the tail gas exiting the bioreactor to provide a substrate having a H₂:CO₂ ratio of 2:1.

9. The method of claim 1 wherein at least a portion of CO₂ and/or H₂ is separated from the tail gas exiting the first bioreactor to provide a substrate having a H₂:CO₂ ratio of 2:1.

10. The method of claim 1 wherein the syngas substrate exiting the gas reformer is sent to a water gas shift module to increase the hydrogen composition of the syngas substrate.

11. The method of claim 1 wherein the tail gas exiting the first bioreactor is sent to a water gas shift module to increase the hydrogen composition of the tail gas stream.

12. The method of claim 1 wherein at least a portion of hydrogen in the syngas substrate is separated from the syngas stream to provide a hydrogen depleted syngas stream and a separated hydrogen stream.

13. The method of claim 12 wherein at least a portion of the separated hydrogen stream is blended with the tail gas stream exiting the first bioreactor to increase the hydrogen composition of the tail gas stream.

14. The method of claim 1 wherein the at least one alcohol produced in the first bioreactor is ethanol.

15. The method of claim 1 wherein the one or more carboxydotrophic microorganisms provided in the first bioreactor is selected from the group consisting of Clostridium autoethanogenum, Clostridium ljungdahlii, Clostridium ragsdalei and Clostridium carboxydivorans.

16. The method of claim 1 wherein the at least one acid produced in the second bioreactor is acetic acid.

17. The method of claim 1 wherein the carboxydotrophic micro-organism in the second bioreactor is Acetobacterium woodii.
